(19) United States
(12) Patent Application Publication
(10) Pub. No.: US 2013/0123610 A1
(43) Pub. Date: May 16, 2013

(54) MRI-COMPATIBLE DEVICE FOR OBTAINING SOFT TISSUE PROPERTIES IN VIVO

(71) Applicants: Michael J. Stebbins, Seattle, WA (US); Peter R. Cavanagh, Seattle, WA (US); Baocheng Chu, Bellevue, WA (US); Michael Fassbind, Seattle, WA (US); David R. Haynor, Seattle, WA (US); William R. Ledoux, Shoreline, WA (US)

(72) Inventors: Michael J. Stebbins, Seattle, WA (US); Peter R. Cavanagh, Seattle, WA (US); Baocheng Chu, Bellevue, WA (US); Michael Fassbind, Seattle, WA (US); David R. Haynor, Seattle, WA (US); William R. Ledoux, Shoreline, WA (US)

(73) Assignee: University of Washington through its Center for Commercialization, Seattle, WA (US)

(21) Appl. No.: 13/486,950

(22) Filed: Jun. 1, 2012

Related U.S. Application Data
(60) Provisional application No. 61/492,686, filed on Jun. 2, 2011.
(60) Provisional application No. 61/650,447, filed on May 22, 2012.

(51) Int. Cl. A61B 5/055

(52) U.S. Cl. CPC A61B 5/055; A61B 5/0555
USPC 600/415; 600/421

(57) ABSTRACT

A method and apparatus for obtaining force versus deformation data for tissue in vivo includes a displacement system that cycles an anatomical member, for example, a foot, repeatedly through a loading/unloading cycle while using a gated imaging procedure such as magnetic resonance imaging to obtain the deformation response of tissues in the foot. The imaging is conducted during the loading/unloading cycle, such that a rate-dependent deformation response is imaged.

### CROSS-REFERENCE TO RELATED APPLICATIONS

This application claims the benefit of Provisional Application No. 61/492,686, filed Jun. 2, 2011, the disclosure of which is hereby incorporated by reference herein. This application also claims the benefit of Provisional Application No. 61/650,447, filed May 22, 2012, the disclosure of which is hereby incorporated by reference herein.

### STATEMENT OF GOVERNMENT LICENSE RIGHTS

This invention was made with Government support under A6973R awarded by Department of Veterans Affairs. The Government has certain rights in the invention.

### BACKGROUND

People with diabetes in the United States currently account for 8.3% of the population, but they undergo over 60% of all non-traumatic lower-limb amputations, or approximately 65,700 amputations per year, according to the Centers for Disease Control and Prevention (CDC). The CDC estimates that if current trends continue, one in three adult Americans will have diabetes by 2050. The epidemic is not contained to the United States; by some estimates the number of people worldwide with diabetes has risen from 30 million in 1985 to 366 million in 2011. In addition to the 4.6 million deaths caused by diabetes in 2011, worldwide diabetes-related healthcare expenditures in that year were estimated at USD 465 billion.

It is estimated that 85% of all non-traumatic lower limb amputations in people with diabetes are preceded by a foot ulcer. Direct costs of diabetic foot ulcerations in 2001 were estimated to be USD 11 billion for the US alone. Despite two recent studies showing a downward trend in diabetic lower limb amputations, with the explosive worldwide growth in new cases of diabetes, foot ulcers and lower limb amputations will continue to be a huge problem for the diabetic population. Developing a better understanding of the pathomechanics behind foot ulcers in people with diabetes and then devising solutions to prevent ulcer formation is of paramount importance to an increasing segment of the world's population.

Diabetes has been shown to increase the stiffness of the plantar soft tissue in cadaveric samples (Pai, 2010). This stiffening would presumably cause shifts in the location and/or magnitude of peak stresses internal to the foot due to the tissue's decreased ability to elastically deform and redistribute pressure under a given load (Gefen, 2003). Experimental observation of in vivo stress distribution internal to the foot is not feasible; instead, computational models are used to understand how changes in soft tissue stiffness might lead to load redistribution, and ultimately, to ulcer formation.

A number of groups have built noteworthy computational foot models. Gefen used a finite element (FE) model to compare loading underneath the first and second metatarsal heads of normal feet and simulated diabetic feet. A review of the literature showed that peak contact stress under the medial metatarsal heads of people with diabetes during standing was approximately 1.5-2.3 times higher than that of normal feet. The FE model showed that for a contact stress increase of 1.5 times, average internal stresses were 4.1 times higher. The author concludes that ulcer formation initiates deep to the plantar surface, most likely under stress risers such as the bony prominences of the metatarsals.

Cheung et al. (2008) have combined an FE foot model with a multi-material orthosis model in order to determine the sensitivity of orthosis design parameters on the reduction of peak plantar contact stress. Using the model and a Taguchi sensitivity analysis (Taguchi, 2005), the group determined that insole stiffness was the second most important factor in reducing peak plantar pressure after the use of an arch-conforming orthosis.

Chen et al. (2010) constructed a detailed three-dimensional (3-D) FE model to study the hypothesis that foot ulceration is initiated internally. The model was validated against an F-Scan plantar pressure measurement with an average difference in plantar pressure predictions under the M2, M3, and M4 metatarsal heads of 14.1%. Under a standing load, the model showed an average internal stress magnification factor of 3.01 under the forefoot using soft tissue material properties from the literature representing normal feet.

An FE model of the first ray of the foot by Budhabhatti et al. (2007) used material properties generated in a separate inverse FE analysis study for the lumped soft tissue. The orientation of the model against a rigid plate and the orientation of the bones to one another were adjusted using an optimization algorithm designed to minimize the error between the model-predicted plantar pressure and experimentally measured pressures. The pressure distribution under the first ray was then calculated for three case studies representing hallux limitus, surgical arthrodesis of the first ray, and a footwear intervention.

The computer models discussed thus far all use lumped-material models for the soft tissue; that is, there is no distinction made between muscle, fat, tendon, etc.

The magnitude and location of peak stresses in the soft tissue of the foot are dependent upon the soft tissue material properties in conjunction with the patient-specific anatomy. Using patient-specific FE models is a means to avoid using averaged tissue material properties and anatomy to represent the large variability inherent to biological tissues. In order to derive patient-specific material properties, an inverse FE analysis can be solved; force and displacement are used as inputs to the model, and an optimization algorithm iterates until it has converged on material properties that satisfy the input conditions to within a user-defined tolerance. Multiple analyses are generally conducted using randomized starting points for the properties to ensure that the converged-upon properties are not dependent upon the starting point (Halloran, 2011).

Previous groups have tested devices capable of generating patient-specific material properties of the foot. Petre et al. (2008) designed a device that was able to apply either a compressive or a shear load to the plantar surface of the forefoot while internal deformations were measured via magnetic resonance imaging (“MRI”). Individual images of the foot were obtained via MRI for five separate loading conditions, from zero to 100% ground reaction force. The resulting properties did not include strain rate dependent effects due to the static loading used. Erdemir et al. (2006) utilized ultrasound imaging and dynamic loading to provide inputs for an inverse FE analysis. They were able to develop hyperelastic material properties of the plantar fat, but the simplified inverse FE model approximated the fat pad as an axi-symmetric, flat disc with a patient-specific thickness. Anatomical boundary conditions for the tissue were not included.

In the present invention, an MRI-compatible, dynamic loading device was developed that uses gated MRI to obtain force versus deformation data. The data obtained with the dynamic loading device is used as inputs to a 3-D FE model to conduct an inverse FE analysis that will solve for patient-specific material properties.

Gated MRI enables static imaging of the dynamically loaded foot, beneficial for both the increased level of safety for test subjects with insensate feet and for the ability to generate viscoelastic material properties. In vivo material stiffness measurements and patient-specific FE models of controls, people with diabetes, people with diabetes and foot deformities, and people with diabetes and a history of ulceration will allow our group to study stress redistribution in the diabetic foot. This will help to increase what is currently known about the pathomechanics of foot ulcer formation in the diabetic population and enable researchers and clinicians to design ulcer preventative measures.

The following references, cited above, are hereby incorporated by reference in their entireties:

Pai, S., and Ledoux, W. R., “The Compressive Mechanical Properties of Diabetic and Nondiabetic Plantar Soft Tissue,” Journal of Biomechanics 43(9):1754-1760, 2010.

Gefen, A., “Plantar Soft Tissue Loading Under the Medial Metatarsals in the Standing Diabetic Foot,” Medical Engineering and Physics 25(6):491-499, 2003.

Cheung, J. T. -M., and Zhang, M., “Parametric Design of Pressure-Relieving Foot Orthosis Using Statistics-Based Finite Element Method”, Medical Engineering and Physics 30(3):269-277, 2008.

Taguchi, G., et al., “Taguchi's Quality Engineering Handbook,” John Wiley & Sons; ASI Consulting Group, Hoboken, N.J.; Livonia, Mich., 2005.

Chen, W. M., et al., “Effects of Internal Stress Concentrations in Plantar Soft-Tissue-a Preliminary Three-Dimensional Finite Element Analysis,” Medical Engineering and Physics 32(4):324-331, 2010.

Budhabhatti S. P., et al., “Finite Element Modeling of the First Ray of the Foot: a Tool for the Design of Interventions,” Journal of Biomechanical Engineering 129(5):750-756, 2007.

Halloran, J. P., and Erdemir, A., “Adaptive Surrogate Modeling for Expedited Estimation of Nonlinear Tissue Properties Through Inverse Finite Element Analysis,” Annals of Biomedical Engineering 39(9): 2388-2397, 2011.

Petre, M., et al., “An MRI-Compatible Foot-Loading Device for Assessment of Internal Tissue Deformation,” Journal of Biomechanics 41(2):470-474, 2008.

Erdemir, A., et al., “An Inverse Finite-Element Model of Heel-Pad Indentation,” Journal of Biomechanics 39(7):1279-1286, 2006.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

A method for obtaining force versus deformation data for tissue in vivo includes securing an anatomical member, for example, a foot, to a fixture that is compatible for use in an imaging apparatus, for example, an MRI scanner, applying a cyclic load to the anatomical member with an actuator configured to apply a cyclic load according to a loading/unloading curve, imaging the anatomical member in a gated mode while applying the cyclic load to obtain images at a predetermined number of locations along the loading/unloading curve over a plurality of cycles, and determining the force applied to the anatomical member at each of the imaged locations.

In an embodiment, the obtained images each include distinguishable images of skin, adipose, and muscle tissue.

In an embodiment, the actuator includes a platen disposed against the anatomical member and driven along a cyclical path that defines the loading/unloading curve, and defining a triangle wave or sinusoidal wave loading/unloading time-displacement path.

In an embodiment, the actuator comprises a slave cylinder that is constructed to be operable within a bore of an MRI scanner during operation, and hydraulically connected to a master cylinder disposed remotely from the MRI scanner.

In an embodiment, the anatomical member is secured to a support fixture that is configured to be inserted into the bore of an MRI scanner.

In another aspect of the invention, a system for obtaining images of an anatomical member using a magnetic resonance imaging (“MRI”) scanner includes a support fixture for restraining and positioning the anatomical member within the bore of the MRI scanner, a displacement system disposed remotely from the MRI scanner, a slave cylinder attached to the support fixture and configured to operate within the bore of the MRI scanner that is operably connected to the master cylinder, and a control system operably connected to drive the master cylinder to drive the slave cylinder along a cyclic path that coordinated with gated imaging operation of the MRI scanner.

In an embodiment, the anatomical member is a foot, and the support fixture includes a cradle assembly configured to receive and restrain a leg attached to the foot.

In an embodiment, the slave cylinder is adjustably attached to the support fixture and includes a platen that is positioned to engage a plantar surface of the foot.

In an embodiment, the displacement system includes a linear actuator that is controlled by the control system to drive the master cylinder along the cyclic path, which may be a triangle wave or sinusoidal wave time-displacement path.

In another aspect of the invention, a system for obtaining force versus displacement data for an anatomical member using an MRI scanner having a bore and operable in a gated mode, includes a support fixture that restrains and positions the anatomical member within the bore of the MRI scanner, a displacement system having a master cylinder disposed remotely from the MRI scanner, a slave cylinder attached to the support fixture and configured to operate within the bore of the MRI scanner to apply loads to the anatomical member, and a conduit operably connecting the master cylinder to the slave cylinder, a pressure sensor operable to measure the forces applied to the anatomical member; and a control system operably connected to drive the master cylinder along a cyclic path that is coordinated with gated imaging operation of the MRI scanner, and to record the measured forces applied to the anatomical member.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a simplified system schematic for a displacement system in accordance with the present invention;

FIG. 2 is an environmental view of a support fixture in accordance with the present invention and configured for use with the displacement system shown in FIG. 1; and

FIG. 3 is a detail view of the support fixture shown in FIG. 2.

### DETAILED DESCRIPTION

The exemplary embodiment of a device and method in accordance with the present invention will now be described, with reference to the figures, wherein like numbers indicate like parts. The exemplary embodiment described herein is not intended to limit the invention, but rather provides persons of skill in the art with a better understanding of the invention.

Methods and systems are disclosed for obtaining in vivo material property information for tissues in an anatomical member, for example, a foot. The material properties obtained in accordance with the present invention reflect viscoelastic or rate-dependent effects resulting from a dynamic loading. In an embodiment, the disclosed method and system obtains force versus deformation data for tissues by applying a known time-dependent loading to a foot in accordance with a design loading/unloading curve, and simultaneously obtaining gated magnetic resonance imaging (“MRI”) data for the foot, at discrete locations along the loading/unloading curve. The gated MRI data is used to calculate deformation of different tissues in the foot at the discrete loading/unloading locations. The loading/unloading curve may be selected to meet the requirements of a particular application. In an exemplary embodiment, the loading/unloading curve approximately uses a constant velocity during the loading phase, and a constant velocity during the unloading phase, i.e., to produce a triangle wave loading/unloading curve. By using the same velocity magnitude for loading and unloading, the obtained imaging data will be consistent for each gated location. In another embodiment, the loading/unloading curve is generally sinusoidal, which was found to reduce the occurrence of pressure waves in the hydraulic system.

The MRI data obtained for each discrete location along the loading/unloading curve are combined, similar to the gated MRI frequently used for cardiac-gated MRI. However, unlike cardiac-gated MRI, in the present system the gated MRI data are obtained at discrete locations based on the externally applied loading, rather than on a physiological trigger. The gated MRI data is obtained over an extended period of time encompassing a plurality of cycles to produce the desired image. The number of cycles is selected to accommodate a particular application, and may depend on the desired image resolution, the volume scanned at each location, and the stroke and frequency of the applied cyclic load. For example, the predetermined gating may be initially triggered by initiation of the application of the load according to the loading/unloading curve, and taken at fixed intervals thereafter wherein the fixed intervals comprise an even factor of the loading/unloading cycle, such that the gating occurs at fixed points along the loading/unloading curve during each cycle. Alternatively, the location of the loading along the loading/unloading curve may be monitored, and used to trigger the MRI gating.

A particular embodiment for use in applying a compressive cyclic load to the plantar surface of a foot will now be described with reference to the FIGURES. Although this exemplary embodiment is directed to a foot and cyclic compressive loading, the method and apparatus may be readily extended to other anatomical members, and to include cyclic shear loading as well as angular displacements, for example, cyclically everting and inverting the ankle joint.

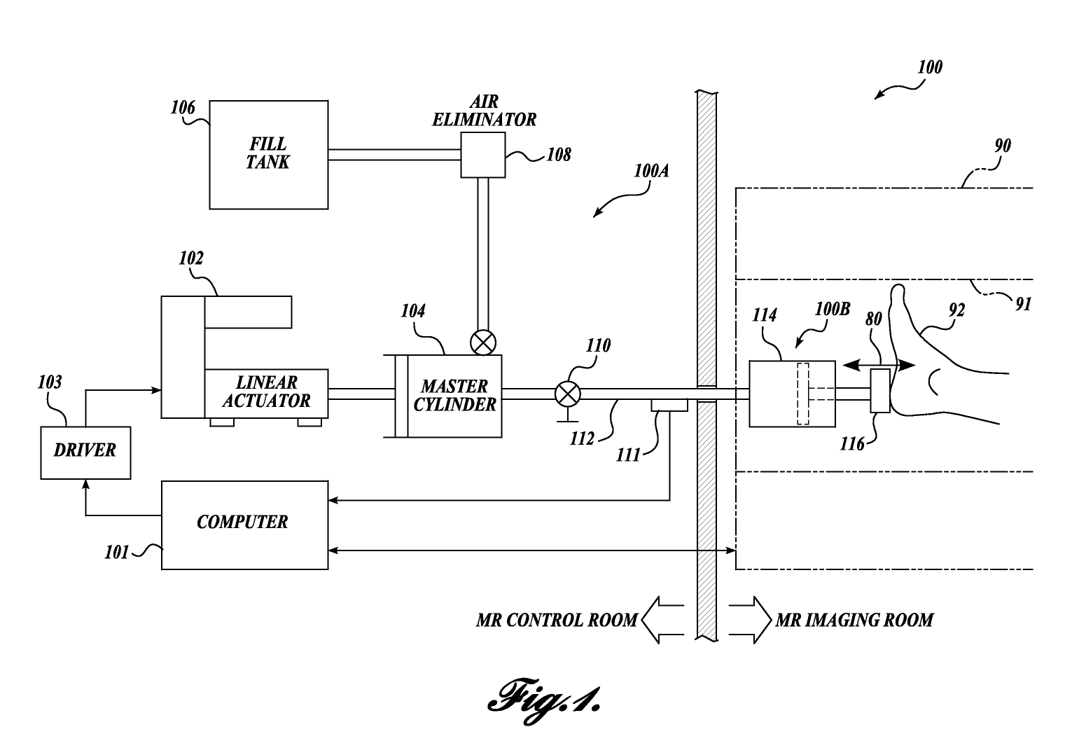

FIG. 1 is a simplified schematic diagram of a displacement system 100 comprising a bifurcated system with an external master actuator subsystem 100A disposed outside of the MRI room (e.g., in the MRI control room), and an MRI-compatible slave subsystem 100B suitable to be placed inside the bore 91 of the MRI scanner 90. The external actuator subsystem 100A in this embodiment includes a stepper motor-linear actuator 102 controlled with a stand-alone stepper driver 103. The linear actuator 102 drives a single-acting aluminum master cylinder 104. Of course, it would be straightforward to implement the displacement system 100 with a double-acting cylinder, which may be preferable in many applications. In the current displacement system 100, the hydraulic fluid is water, which was selected for its ease of use, and to simplify assembly and disassembly of the displacement system 100. However, other conventional hydraulic fluids are contemplated, for example, a mineral oil or the like. An oil-based hydraulic fluid will be preferred in many applications. The hydraulic fluid, which in FIG. 1 is provided to the master cylinder 104 from a fill tank 106, is processed to remove dissolved gasses by passing the fluid through a conventional hydronic heating air eliminator 108.

The master cylinder 104 is connected to a slave cylinder 114 through a suitable conduit 112. In the present embodiment the conduit 112 comprises about nine meters of 9.65 mm I.D. vacuum-rated nylon tubing. The conduit 112 extends through a wall port into the MRI room. Vacuum-rated tubing was selected due to the system undergoing a small negative pressure during the unloading portion of the cycle. A check valve 110 is provided to guard against over-pressurization. The slave cylinder 114 piston is fixed to a platen 116 that is configured to engage the subject's foot 92, as discussed below. The slave cylinder 114 is made from materials suitable for use in the bore 91 of the MRI scanner 90. Preferably, the slave cylinder 114 is made substantially or entirely from non-metallic materials. As indicated by the arrow 80, the platen 116 is oscillated to apply a cyclic compressive load to the subject's foot 92.

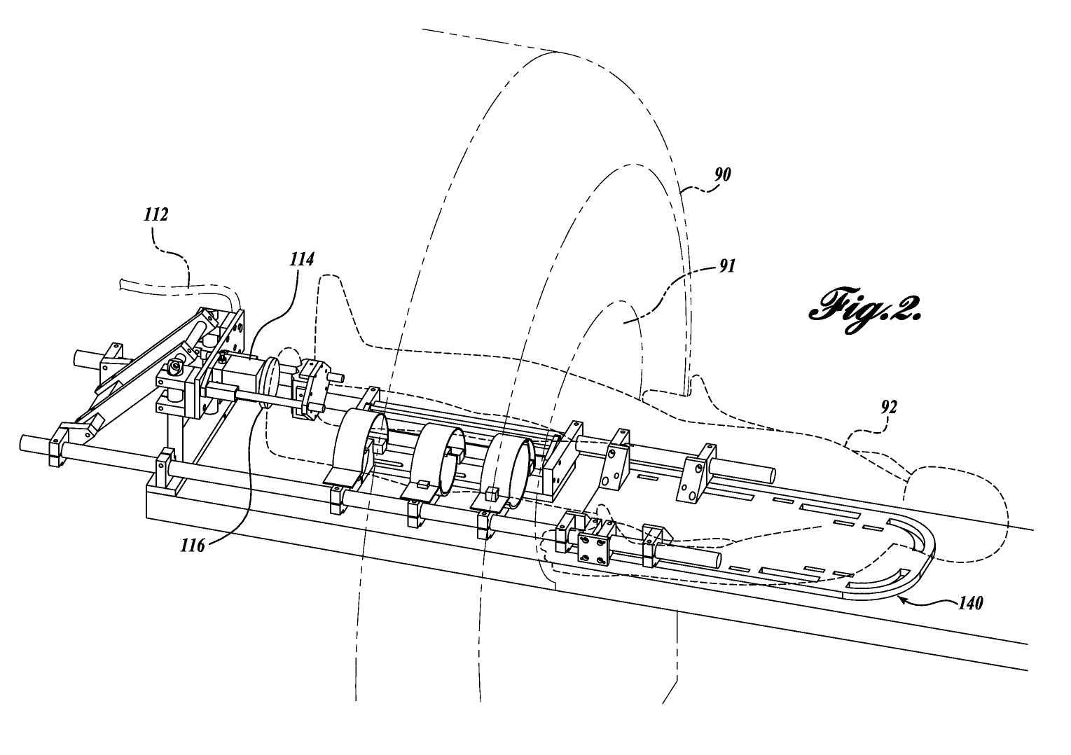

FIG. 2 shows the subject 92 supported on an MRI-compatible support fixture 140, with the subject 92 positioned at least partially within the bore 91 of the MRI scanner 90. The slave cylinder 114 and loading platen 116 assembly is adjustably attached to the support fixture 140. The fixture 140 restrains the leg and the foot of the subject 92 in the desired position, and such that the subject's foot 92 engages the platen 116. Preferably, the fixture 140 also restrains the subject's torso in order to minimize movement.

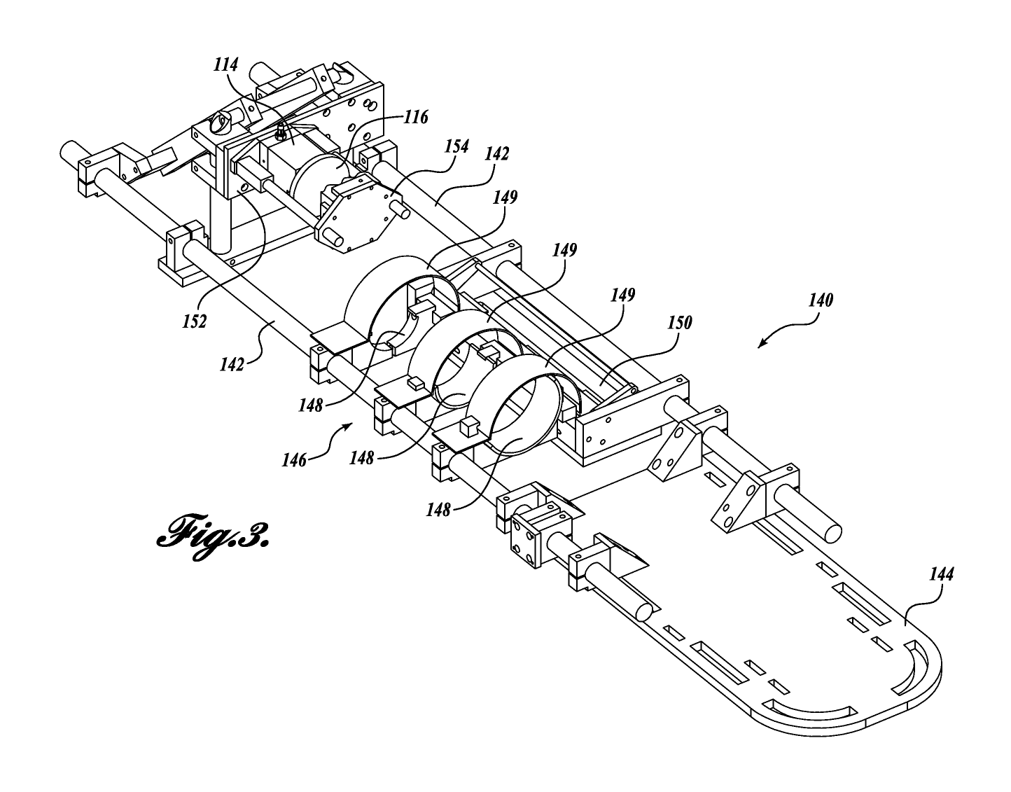

A perspective view of the support fixture 140 is shown in FIG. 3. The fixture 140 comprises two parallel cylindrical rails 142. A backrest 144 is adjustably mounted to the rails 142 near a proximal end, and is configured to be adjustable longitudinally to accommodate different subjects.

A leg restraint 146 is adjustably attached along a mid-portion of the rails 142. In this embodiment, the leg restraint 146 includes three longitudinally spaced cradle assemblies 148 that receive the subject's leg. The cradle assemblies 148 preferably include a foam cover (not shown) for comfort, and further include webbing or straps 149 that are tightened over the subject's leg, to restrain the leg in a desired position. Typically, the cradle assemblies 148 are adjusted longitudinally such that two of the cradle assemblies 148 engage the lower leg of the subject, and the third cradle assembly 148 engages the upper leg. A cam mechanism 150 provides for an easy securement and release of the cradle assemblies 148. The cam mechanism 150 is designed to allow for minute adjustments for the comfort of the subject 92. In this embodiment the leg restraint 146 and cam mechanism 150 may be reversed (medially/laterally) to secure either the right leg or the left leg of the subject 92.

The slave cylinder 114 is adjustably attached near the distal end of the rails 142 on an upright support structure 152. The slave cylinder 114 is positioned generally in alignment with the leg restraint 146, and is adjustable in the anterior/posterior direction, e.g., to align with either the hindfoot or forefoot. The slave cylinder 114 is also adjustable in the medial/lateral direction, e.g., for use with the left or right foot. A removable and adjustable forefoot support 154 is attached to the upright support structure 152 and positionable over the top of the subject's foot. The forefoot support 154 is used when the subject's forefoot is tested to prevent dorsiflexion of the ankle when the cyclic load is applied by the platen 116.

To further minimize motion during testing, in the current embodiment, the subject's torso is also held in place with adjustable straps (not shown) that extend over the shoulders and around the waist of the subject and connect to the backrest 144. The components of the current support fixture 140 are modular to allow for subject-specific adjustments and for easy disassembly and storage. For MRI-compatibility, in a current embodiment all components of the support fixture 140 are either machined polycarbonate or acetal plastics, or are nylon, fiberglass-reinforced nylon, polypropylene, polyethylene or particle board hardware. In a preferred embodiment, the support fixture 140, slave cylinder 114, platen 116, and related components that extend into the MRI room are entirely or substantially non-metal.

Referring again to FIG. 1, the stepper driver 103 for the linear actuator 102 is controlled by manufacturer-provided software. Application-specific data acquisition software was developed using the LabVIEW® software package to acquire and log data, and to send displacement-synchronized trigger signals to the MRI control center. The software runs on a laptop computer 101 (Intel Pentium M, 1.6 GHz, 2.0 GB RAM), which hosts an external data acquisition board (Model: USB-6212, 400 kS/s, 16-bits, National Instruments, Austin, Tex.). All signals are acquired at 2500 Hz to allow for digital smoothing in post-processing. The position of the linear actuator 102 versus time is determined from a rotary encoder (resolution=0.18° of stepper motor rotation or 0.0008 mm of actuator displacement) affixed to the stepper motor. The load applied to the foot is measured via a pressure transducer 111 (Model: PX209-200, Omega Engineering, Stamford, Conn.) installed in the hydraulic system.

The pressure data is calibrated to account for frictional losses and the compressibility of any air remaining in the fluid after bleeding. Calibration is achieved via testing while simultaneously acquiring hydraulic fluid pressure data and the force being transmitted by the loading platen. During bench top testing, a 2224 N loadcell (Model: MC3A-1000, Advanced Mechanical Technology, Inc., Watertown, Mass.) was placed in series with the platen and the loading apparatus. With these data, the actual load on the foot along with the appropriate temporal shift can be determined. Verification testing of the system is described in some detail in the priority U.S. Provisional Application No. 61/650,447, which is incorporated by reference above.

In one embodiment, a gated MRI-protocol is used to obtain 16 static 3-D images of the foot while the loading platen translates dynamically from zero to the patient-specific maximum displacement and then back to zero repeatedly. In conventional cardiac gating, a physiological signal from the patient, for example, a heart beat signal derived from an ECG, is used to trigger the MRI scanner to acquire all MRI signals at a time when the position of the dynamically displacing heart is the same. Only objects with a periodic or quasi-periodic motion can be imaged in this manner. In an exemplary gated-imaging system, the MRI Control and Data Acquisition System (CDAS) is triggered by the Basic Triggering Unit (BTU), which converts various analog physiologic signals into a digital data stream.

The MRI images obtained show and distinguish skin tissue, adipose tissue, and muscle tissue; therefore, the images can be used to determine the individual displacement of each of these tissues resulting from the applied cyclic load.

For use with the system shown in FIGS. 1 and 2, the BTU is replaced by data that mimics a square wave peripheral pulse unit (PPU) signal. A simulated PPU pulse is generated and sent once per loading cycle by LabVIEW via an RS-232 serial port and is then converted to a fiber optic signal via an RS-232-to-HP Versalink® fiber optic converter

(Electro Standards Laboratories, Cranston, R.I.) to interface with the CDAS. In addition, five separate status messages are included with the PPU signal every 20 milliseconds. An Achieva™ 3.0T MRI system 90 was used for all MRI testing.

The displacement system 100 incorporates several redundant safety measures to protect the subject from over-loading and/or painful loading. An electronic, solenoid-operated hydraulic valve is installed in the hydraulic system. When power is removed from the solenoid, the valve opens and the pressurized hydraulic fluid exits the system into a waste container, thereby removing load from the loading platen. Power to the solenoid can be removed by any of the following: 1) an emergency stop (E-stop) button near the test operator, 2) an E-stop button at the test subject's side inside the MRI, 3) by the system software, if a patient-specific not-to-exceed pressure is exceeded, and 4) by a virtual button on the LabVIEW front panel.

The not-to-exceed pressure is the system pressure at the subject's ground reaction force increased by a factor of 1.2 to account for pressure surges. The E-stop button inside the MRI is fiber-optic and interfaces with a controller (both from Banner Engineering, Minneapolis, Minn.) inside the MRI control room, and is mountable to allow for left or right hand access. The button was modified so as to remove all metal inside of it except for several small hardware pieces, and is securely mounted to the loading frame near one of the test subject's hands. The hydraulic system also includes an adjustable mechanical pressure relief valve. This valve is set to release any pressure greater than the patient-specific not-to-exceed pressure. As a final measure, the master piston is positioned in the master cylinder such that if it were to extend past a failed electronic limit switch, it could travel less than 1 mm before contacting the rigid, aluminum cylinder bottom. The 1 mm buffer is a result of setting the location of the electronic limit switch by hand so as not to accidentally bottom-out the piston during cycling, which could damage the actuator.

The system shown in FIGS. 1-3 is configured for use with an MRI scanner 90, operated in a gated mode while a cyclic force or loading is applied to an anatomical member, for example a subject's foot. The MRI scanner is configured to obtain image data at predetermined positions on the loading/unloading curve over a number of loading/unloading cycles, and while monitoring the force or loading applied to the anatomical member. Therefore, the disclosed system is uniquely able to obtain force versus deformation data for the anatomical member undergoing a dynamic or time-varying force, such that rate of deformation effects are included.

These data may then be used, for example, as inputs in conjunction with existing 3-D FE models to conduct an inverse FE analysis that will solve for patient-specific material properties that include rate of strain effects.

In a particular example, MRI imaging was performed on the 3.0T Philips Achieva MRI system 90 on a test subject. High-resolution, static images of the subject's foot were obtained prior to dynamic loading to create the unloaded soft tissue geometry for the FEA model. Bone tissue geometry for this test subject was obtained from previously collected CT images.

Scanning was performed with the loading device 100 cycling at 0.2 Hz and the MRI system imaging at a 100% duty cycle. This enabled the MRI system to obtain all of the images pertaining to the loading portion of the loading/unloading cycle in one scan sequence, and then collect all of the images pertaining to the unloading portion of the cycle in a subsequent scan sequence. Voxel size was 1.0 mm³, and image data were obtained at 12 locations along the loading/unloading curve. These parameters allowed the group to successfully complete a dynamic scan in approximately 11 minutes.

A T1-weighted, 3-D FFE sequence was used to obtain 70 transverse slices of the heel, with each slice having a thickness of 2 mm with a slice spacing of 1 mm. A volume equal to 92×56×70m (A/P×M/L×S/I) was imaged with a voxel size of 1.0 mm³ (ET: 36, TR: 11.4, TE: 5.0, Frequency: 128, Phase: 89, Flip: 35). The trigger delay, a required input responsible for setting the delay between the PPU trigger signal being received by the MRI Control and Data Acquisition System (CDAS) and the imaging occurring, was 152 milliseconds.

During imaging, the loading device actuator was set to displace with a sine wave displacement profile at 0.2 Hz and peak amplitude of 15.625 mm. In the test system, an actuator displacement of 15.625 mm would result in a loading platen displacement of approximately 14.22 mm. Hydraulic system pressure data and actuator encoder data were acquired during testing at a 2500 Hz sampling rate.

One stack of T1-weighted high-resolution images of the unloaded foot was acquired in a scan lasting approximately 31 minutes. Another stack of T2-weighted high-resolution images of the unloaded foot was acquired in a scan lasting approximately 15 minutes. These first two stacks were static and imaged the entire foot. Twelve stacks of images of the dynamically-loaded foot were obtained and processed using the gated protocol discussed above, and included only a portion of the foot. Compression of the soft tissue and the tissue's return to an unloaded state can clearly be witnessed during the 12 locations along the loading/unloading curve for which image data was obtained.

Due to the relative thickness of the skin and low resolution of the dynamic images, there was significant variability in the calculated thickness of the skin between three separate trials. The average value of the thickness of the plantar skin varied from a maximum of 3.4 mm to a minimum of 2.7 mm. The average value of the plantar fat pad thickness varied from a maximum of 16.0 mm to a minimum of 10.2 mm.

The calculated strain in the plantar skin shows a large range within one standard deviation from the average of the three trials. The calculated strain in the plantar fat pad shows a maximum strain of 36.3% compressive strain occurring at the sixth of 12 locations along the loading/unloading curve for which image data was obtained.

Derived load on the platen showed a maximum of 141.8 N occurring at the seventh location. Derived load on the platen versus total average displacement of the soft tissue was calculated, as was the stiffness of the soft tissue in the high-stiffness region of the curve. The stiffness was calculated to be 55 N/mm.

The above described procedure and results are representative, and it is contemplated that the various test parameters will be selected to accommodate a particular application. Selection of suitable parameters is believed to be within the skill in the art. In particular, it is expressly contemplated that the applied loading may be applied at a higher frequency to more closely correspond to a typical walking gait. For example, the frequency may be in the range of 0.1 to 10 Hz.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for obtaining force versus deformation data for a tissue in vivo in an anatomical member, comprising:
securing the anatomical member to a fixture that is configured to be compatible for use in an imaging apparatus,
applying a cyclical load to the anatomical member with an actuator that is configured to apply and release a load on the anatomical member according to a loading/unloading curve;
operating the imaging apparatus in a gated mode while applying the cyclical load to obtain images of the anatomical member at a plurality of predetermined and fixed locations along the loading/unloading curve, wherein the images are obtained at the plurality of locations using data obtained over a plurality of cycles through the loading/unloading curve; and
determining a force applied to the anatomical member for each of the plurality of predetermined and fixed locations along the loading/unloading curve.

2. The method of claim 1, wherein the anatomical member comprises a foot.

3. The method of claim 1, wherein the imaging apparatus comprises a magnetic resonance imaging scanner.

4. The method of claim 1, wherein the images include distinguishable images of skin, adipose, and muscle tissues within the anatomical member.

5. The method of claim 1, wherein the actuator comprises a platen disposed to abut the anatomical member and driven along a cyclical path to apply the cyclical load to the anatomical member.

6. The method of claim 1, wherein the loading/unloading curve comprises a triangle wave or a sine wave time-displacement path.

7. The method of claim 3, wherein the actuator comprises a slave cylinder that is constructed to be compatible with operation within a bore of the magnetic resonance imaging scanner during operation.

8. The method of claim 7, wherein the slave cylinder is constructed solely from non-metallic components.

9. The method of claim 7, wherein the actuator further comprises a master cylinder that is hydraulically connected to the slave cylinder with a conduit, wherein the master cylinder is disposed remotely from the magnetic resonance imaging scanner.

10. The method of claim 3, wherein the anatomical member is secured to a support fixture that is configured to be inserted into a bore of the magnetic resonance imaging scanner.

11. A system for obtaining images of an anatomical member using a magnetic resonance imaging (“MRI”) scanner having a bore and operable to obtain gated images of the anatomical member, the system comprising:
a support fixture configured to restrain the anatomical member and to position the anatomical member within the bore of the MRI scanner;
a displacement system having a master cylinder disposed remotely from the MRI scanner, a slave cylinder attached to the support fixture and configured to operate within the bore of the MRI scanner to apply loads to the anatomical member, and a conduit operably connecting the master cylinder to the slave cylinder; and
a control system operably connected to drive the master cylinder such that the slave cylinder is displaced along a cyclic path that is coordinated with gated imaging operation of the MRI scanner.

12. The system of claim 11, wherein the anatomical member comprises a foot.

13. The system of claim 12, wherein the support fixture comprises a cradle assembly configured to receive and restrain a leg attached to the foot.

14. The system of claim 12, wherein the slave cylinder further comprises a platen that is positioned to engage a plantar surface of the foot.

15. The system of claim 14, wherein the slave cylinder is adjustably attached to the support fixture such that the slave cylinder can be attached at a first position wherein the platen engages a forefoot portion of the foot, and the slave cylinder can be attached at a second position wherein the platen engages a hindfoot portion of the foot.

16. The system of claim 14, wherein the slave cylinder is adjustably attached to the support fixture such that the slave cylinder can be selectively attached on a left side of the support fixture and can be selectively attached on a right side of the support fixture.

17. The system of claim 12, wherein the displacement system further comprises a linear actuator that is controlled by the control system to drive the master cylinder along the cyclic path.

18. The system of claim 12, wherein the displacement system applies a continuously varying compressive force on the foot while the MRI scanner is operated to obtain gated images of the foot.

19. A system for obtaining force versus displacement data for an anatomical member using a magnetic resonance imaging (“MRI”) scanner having a bore and operable in a gated mode, the system comprising:
a support fixture configured to restrain the anatomical member and to position the anatomical member within the bore of the MRI scanner;
a displacement system having a master cylinder disposed remotely from the MRI scanner, a slave cylinder attached to the support fixture and configured to operate within the bore of the MRI scanner to apply loads to the anatomical member, and a conduit operably connecting the master cylinder to the slave cylinder;
a pressure sensor operable to measure the forces applied to the anatomical member; and
a control system operably connected to drive the master cylinder along a cyclic path that is coordinated with gated imaging operation of the MRI scanner, and to record the measured forces applied to the anatomical member.

20. The system of claim 19, wherein the anatomical member comprises a foot.

21. The system of claim 20, wherein the support fixture comprises a cradle assembly configured to receive and restrain a leg attached to the foot.

22. The system of claim 20, wherein the slave cylinder further comprises a platen that is positioned to engage a plantar surface of the foot.

23. The system of claim 22, wherein the slave cylinder is adjustably attached to the support fixture such that the slave cylinder can be attached at a first position wherein the platen engages a forefoot portion of the foot, and the slave cylinder can be attached at a second position wherein the platen engages a hindfoot portion of the foot.

24. The system of claim 19, wherein the pressure sensor measures the force applied to the anatomical member by measuring the fluid pressure provided to the slave cylinder.
